(19) United States
(12) Patent Application Publication
(10) Pub. No.: US 2005/0070468 A1
(43) Pub. Date: Mar. 31, 2005

(54) Use of fk506 and analogues for treating allergic diseases

(75) Inventor: Ryuji Ueno, Montgomery, MD (US)

Correspondence Address: OBLON, SPIVAK, MCCLELLAND, MAIER & NEUSTADT, P.C., 1940 DUKE STREET, ALEXANDRIA, VA, 22314, US

(73) Assignee: SUCAMPO AG, ZUG (CH)

(21) Appl. No.: 10/495,425

(22) PCT Filed: Nov. 20, 2002
(86) PCT No.: PCT/JP02/12096

Related U.S. Application Data
(60) Provisional application No. 60/331,722, filed on Nov. 21, 2001.

(51) Int. Cl. A61K 38/13; A61K 31/4745

(52) U.S. Cl. 514/11; 514/291

(57) ABSTRACT

The present invention provides, in the treatment of allergic diseases using an interleukin 2 inhibitor, particularly a macrolide compound such as FK506, a method of treating an allergic disease, which includes setting a leading period for pre-administration of an interleukin 2 inhibitor.

This application is based on application No. 60/331,722 filed in United States of America, the content of which is incorporated hereinto by reference.

### TECHNICAL FIELD OF THE INVENTION

The present invention relates to a method for treating allergic diseases.

### BACKGROUND ART

When a foreign matter invades the body, an antibody or sensitized lymphocyte is generated by an immune response. The antibody or sensitized lymphocyte reacts with the foreign matter when it invades again, whereby the foreign matter is removed or attenuated. This is the so-called “immunity”. When the reaction proceeds inversely to damage the body, it is called an “allergy”.

An allergic reaction was classified into IgE dependent anaphylactic type (I type), cytotoxic type (II type), immune complex type (III type), and cellular immunity type (IV type) by Coombs and Gell (1963) based on the mechanism of immune reaction. It is considered that these reaction types are involved in a complicated manner to cause allergic diseases in the living body.

The I type allergy is a general name for hypersensitiveness caused by the reaction with IgE antibody upon contact with an allergen, and this type is also called an atopic disease. For example, bronchial asthma, allergic rhinitis, allergic conjunctivitis, atopic dermatitis, food allergy, and a part of drug allergy fall under the atopic diseases.

In the meantime, a macrolide compound, such as FK506, and cyclosporins are known to be effective for the treatment of allergic diseases such as allergic conjunctivitis, spring catarrh, atopic dermatitis and the like (WO 92/19278 etc.).

### DISCLOSURE OF THE INVENTION

The present inventor has conducted intensive studies and surprisingly found that, in the treatment of allergic diseases using an interleukin 2 (hereinafter sometimes referred to simply as IL-2) inhibitor, expression of the effect is drastically increased by setting a leading period for pre-administration of an IL-2 inhibitor, which resulted in the completion of the present invention.

Accordingly, the present invention provides the following.
(1) A pharmaceutical agent for pre-administration, which comprises an interleukin 2 inhibitor (IL-2 inhibitor) as an active ingredient, and which is used for treating an allergic disease, wherein the treatment includes a leading period for pre-administration of the IL-2 inhibitor to a subject in need of the treatment of allergic disease, and administration of an effective amount of an IL-2 inhibitor.

(2) The pharmaceutical agent of (1), wherein the IL-2 inhibitor is a macrolide compound or a cyclosporin.

(3) The pharmaceutical agent of (2), wherein the macrolide compound is a tricyclo compound (I) of the following formula (hereinafter sometimes referred to simply as tricyclo compound (I)); 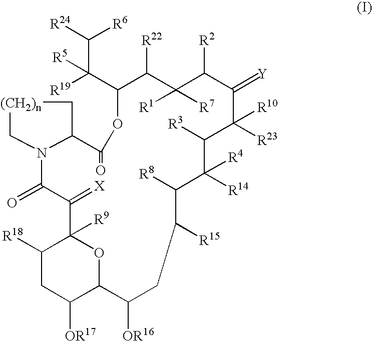
wherein adjacent pairs of R¹ and R², R³ and R⁴, and R⁵ and R⁶ each independently
a) consist of two adjacent hydrogen atoms, wherein R² is optionally alkyl, or
b) form another bond between carbon atoms binding with the members of each pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkenyl, alkyl substituted by one or more hydroxy, alkenyl substituted by one or more hydroxy, or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

In addition to the meaning noted above, Y, R¹⁰ and R²³ may form, together with the carbon atom they bind with, a saturated or unsaturated 5 or 6-membered heterocyclic group containing nitrogen atom, sulfur atom and/or oxygen atom, wherein the heterocyclic group may be substituted by one or more group(s) selected from the group consisting of alkyl, hydroxy, alkyloxy, benzyl, a group of the formula —CH₂Se(C₆H₅), and alkyl substituted by one or more hydroxy, or a pharmaceutically acceptable salt thereof.
(4) The pharmaceutical agent of (2) or (3), wherein the macrolide compound is FK506.
(5) The pharmaceutical agent of (1), wherein the IL-2 inhibitor is a preparation for local administration, especially a preparation for local administration to the eye or the nose.
(6) The pharmaceutical agent of (1), wherein the allergic disease is allergic conjunctivitis.
(7) The pharmaceutical agent of (1), wherein the allergic disease is seasonal allergic disease, especially seasonal allergic conjunctivitis.
(8) A pharmaceutical composition for pre-administration, which comprises an IL-2 inhibitor as an active ingredient and a pharmaceutically acceptable carrier, and which is used for treating an allergic disease, wherein the treatment includes a leading period for pre-administration of the IL-2 inhibitor to a subject in need of the treatment of allergic disease, and administration of an effective amount of an IL-2 inhibitor.
(9) The pharmaceutical composition of (8), wherein the IL-2 inhibitor is a macrolide compound or a cyclosporin.
(10) The pharmaceutical composition of (9), wherein the macrolide compound is a tricyclo compound (I), or a pharmaceutically acceptable salt thereof.
(11) The pharmaceutical composition of (9) or (10), wherein the macrolide compound is FK506.
(12) The pharmaceutical composition of (8), wherein the IL-2 inhibitor is a preparation for local administration, especially a preparation for local administration to the eye or the nose.
(13) The pharmaceutical composition of (8), wherein the allergic disease is allergic conjunctivitis.
(14) The pharmaceutical composition of (8), wherein the allergic disease is seasonal allergic disease, especially seasonal allergic conjunctivitis.
(15) A method for treating an allergic disease, which comprises pre-administering an IL-2 inhibitor for a leading period and then administering an effective amount of an IL-2 inhibitor to a subject in need of a treatment of an allergic disease.
(16) The method of (15), wherein the IL-2 inhibitor is a macrolide compound or a cyclosporin.
(17) The method of (16), wherein the macrolide compound is a tricyclo compound (I), or a pharmaceutically acceptable salt thereof.
(18) The method of (16) or (17), wherein the macrolide compound is FK506.
(19) The method of (15), wherein the IL-2 inhibitor is a preparation for local administration, especially a preparation for local administration to the eye or to the nose.
(20) The method of (15), wherein the allergic disease is allergic conjunctivitis.
(21) The method of (15), wherein the allergic disease is a seasonal allergic disease, especially seasonal allergic conjunctivitis.
(22) Use of an IL-2 inhibitor for the production of a pharmaceutical composition for pre-administration, which comprises the IL-2 inhibitor as an active ingredient and a pharmaceutically acceptable carrier, and which is used for treating an allergic disease, wherein the treatment includes a leading period for pre-administration of the IL-2 inhibitor to a subject in need of the treatment of allergic disease, and administration of an effective amount of an IL-2 inhibitor.
(23) The use of (22), wherein the IL-2 inhibitor is a macrolide compound or a cyclosporin.
(24) The use of (23), wherein the macrolide compound is a tricyclo compound (I), or a pharmaceutically acceptable salt thereof.
(25) The use of (23) or (24), wherein the macrolide compound is FK506.
(26) The use of (22), wherein the IL-2 inhibitor is a preparation for local administration, especially a preparation for local administration to the eye or the nose.
(27) The use of (22), wherein the allergic disease is allergic conjunctivitis.
(28) The use of (22), wherein the allergic disease is seasonal allergic disease, especially seasonal allergic conjunctivitis.
(29) A commercial package comprising the pharmaceutical composition of any of the above-mentioned (8) to (14) and a written matter associated therewith, the written matter stating that the pharmaceutical composition can or should be used for pre-administration for treating allergic diseases, wherein the treatment includes a leading period for pre-administration of the IL-2 inhibitor to a subject in need of the treatment of allergic disease, and administration of an effective amount of an IL-2 inhibitor.

### DETAILED DESCRIPTION OF THE INVENTION

The IL-2 inhibitor to be used in the present invention is not particularly limited and may be any as long as it has an IL-2 inhibitory activity. One example thereof is an IL-2 production inhibitor. Another example is an IL-2 signal transduction inhibitor. Preferable examples thereof include macrolide compounds such as FK506, Ascomycin derivative, Rapamycin derivative and the like, and cyclosporins and the like.

Specific examples of macrolide compound include tricyclo compound (I) of the following formula and a pharmaceutically acceptable salt thereof. 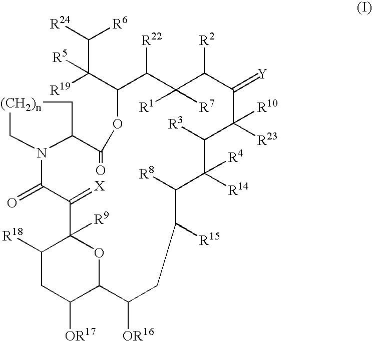
wherein adjacent pairs of R¹ and R², R³ and R⁴, and R⁵ and R⁶ each independently
a) consist of two adjacent hydrogen atoms, wherein R² is optionally alkyl, or
b) form another bond between carbon atoms binding with the members of each pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkenyl, alkyl substituted by one or more hydroxy, alkenyl substituted by one or more hydroxy or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

In addition to the meaning noted above, Y, R¹⁰ and R²³ may form, together with the carbon atom they bind with, a saturated or unsaturated 5 or 6-membered heterocyclic group containing nitrogen atom, sulfur atom and/or oxygen atom, wherein the heterocyclic group may be substituted by one or more group(s) selected from alkyl, hydroxy, alkyloxy, benzyl, a group of the formula —CH₂Se(C₆H₅), and alkyl substituted by one or more hydroxy.

Preferable R²⁴ is, for example, cyclo(C₅-C₇)alkyl optionally having suitable substituent, such as the following.
(a) 3,4-dioxocyclohexyl,
(b) 3-R²⁰-4-R²¹-cyclohexyl,
   wherein R²⁰ is hydroxy, alkyloxy or —OCH₂OCH₂CH₂OCH₃, and
   R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy, or R²⁵R²⁶CHCOO— (wherein R²⁵ is hydroxy optionally protected where desired or protected amino, and R²⁶ is hydrogen atom or methyl) or R²⁰ and R²¹ in combination form an oxygen atom of epoxide ring, and
(c) cyclopentyl wherein cyclopentyl is substituted by methoxymethyl, protected hydroxymethyl where desired, acyloxymethyl (wherein acyl moiety is optionally quaternized dimethylamino where desired or optionally esterified carboxy), one or more optionally protected amino and/or hydroxy, or aminooxalyloxymethyl. Preferable example includes 2-formyl-cyclopentyl.

The definition of each symbol used in the formula (I), specific examples thereof and preferable embodiments thereof are explained in detail in the following.

“Lower” means that a group has 1 to 6 carbon atoms unless otherwise indicated.

Preferable examples of the alkyl moiety of “alkyl” and “alkyloxy” include linear or branched aliphatic hydrocarbon residue, such as lower alkyl (e.g., methyl, ethyl, propyl, isopropyl, butyl, isobutyl, pentyl, neopentyl, hexyl and the like).

Preferable examples of “alkenyl” include linear or branched aliphatic hydrocarbon residue having one double bond, such as lower alkenyl (e.g., vinyl, propenyl (e.g., allyl and the like), butenyl, methylpropenyl, pentenyl, hexenyl and the like).

Preferable examples of “aryl” include phenyl, tolyl, xylyl, cumenyl, mesityl, naphthyl and the like.

Preferable examples of the protective group for “protected hydroxy” and “protected amino” include 1-(lower alkylthio) (lower)alkyl such as lower alkylthiomethyl (e.g., methylthiomethyl, ethylthiomethyl, propylthiomethyl, isopropylthiomethyl, butylthiomethyl, isobutylthiomethyl, hexylthiomethyl and the like), with more preference given to C₁-C₄ alkylthiomethyl and most preference given to methylthiomethyl;
tri-substituted silyl such as tri(lower)alkylsilyl (e.g., trimethylsilyl, triethylsilyl, tributylsilyl, tert-butyl dimethylsilyl, tri-tert-butylsilyl and the like), and lower alkyldiarylsilyl (e.g., methyldiphenylsilyl, ethyldiphenylsilyl, propyldiphenylsilyl, tert-butyldiphenylsilyl and the like), with more preference given to tri(C₁-C₄)alkylsilyl and C₁-C₄ alkyldiphenylsilyl, and most prefererence given to tert-butyl-dimethylsilyl, tert-butyldiphenylsilyl;
acyl such as aliphatic acyl, aromatic acyl and aliphatic acyl substituted by aromatic group, which are derived from carboxylic acid, sulfonic acid and carbamic acid; and the like.

The aliphatic acyl is exemplified by lower alkanoyl optionally having one or more suitable substituent(s) (e.g., carboxy) such as formyl, acetyl, propionyl, butyryl, isobutyryl, valeryl, isovaleryl, pivaloyl, hexanoyl, carboxyacetyl, carboxypropionyl, carboxybutyryl, carboxyhexanoyl and the like;
cyclo(lower)alkyloxy(lower)alkanoyl optionally having one or more suitable substituent(s) (e.g., lower alkyl) such as cyclopropyloxyacetyl, cyclobutyloxypropionyl, cycloheptyloxybutyryl, mentyloxyacetyl, mentyloxypropionyl, mentyloxybutyryl, mentyloxypentanoyl, mentyloxyhexanoyl and the like,
camphorsulfonyl;
lower alkylcarbamoyl having one or more suitable substituent(s) such as carboxy, protected carboxy and the like, such as carboxy(lower)alkylcarbamoyl (e.g., carboxymethylcarbamoyl, carboxyethylcarbamoyl, carboxypropylcarbamoyl, carboxybutylcarbamoyl, carboxypentylcarbamoyl, carboxyhexylcarbamoyl) and
tri(lower)alkylsilyl(lower)alkyloxycarbonyl(lower)-alkylcarbamoyl (e.g., trimethylsilylmethoxycarbonylethylcarbamoyl, trimethylsilylethoxycarbonylpropylcarbamoyl, triethylsilylethoxycarbonylpropylcarbamoyl, tert-butyl dimethylsilylethoxycarbonylpropylcarbamoyl, trimethylsilylpropoxycarbonylbutylcarbamoyl).

Aromatic acyl is exemplified by aroyl optionally having suitable substituent(s) (e.g., nitro), such as benzoyl, toluoyl, xyloyl, naphthoyl, nitrobenzoyl, dinitrobenzoyl, nitronaphthoyl and the like; and arenesulfonyl optionally having one or more suitable substituent(s) (e.g., halogen), such as benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl and the like.

The aliphatic acyl substituted by aromatic group may be, for example, ar(lower)alkanoyl optionally having one or more suitable substituent(s) (e.g., lower alkyloxy, trihalo(lower)alkyl and the like), wherein specific examples are phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl and the like.

Of the above-mentioned acyl, more preferable acyl includes C₁-C₄ alkanoyl optionally having carboxy, cyclo(C₅-C₆)alkyloxy(C₁-C₄)alkanoyl having two (C₁-C₄)alkyl in the cycloalkyl moiety, camphorsulfonyl, carboxy(C₁-C₄)alkylcarbamoyl, tri(C₁-C₄)alkylsilyl(C₁-C₄)alkyloxycarbonyl(C₁-C₄)alkylcarbamoyl, benzoyl optionally having 1 or 2 nitro groups, and benzenesulfonyl having halogen, phenyl(C₁-C₄)alkanoyl having C₁-C₄ alkyloxy and trihalo(C₁-C₄)alkyl. Of these, most preferred are acetyl, carboxypropionyl, mentyloxyacetyl, camphorsulfonyl, benzoyl, nitrobenzoyl, dinitrobenzoyl, iodobenzenesulfonyl, 2-trifluoromethyl-2-methoxy-2-phenylacetyl and the like.

Preferable examples of the “heterocyclic group consisting of saturated or unsaturated 5 or 6-membered ring having nitrogen atom, sulfur atom and/or oxygen atom” are pyrolyl, tetrahydrofuryl and the like.

The “heteroaryl optionally having a suitable substituents” moiety of the “heteroaryloxy optionally having a suitable substituent” is that exemplified for R¹ of the compound of the formula I of EP-A-532088, with preference given to 1-hydroxyethylindol-5-yl. This publication is incorporated hereinto by reference.

The tricyclo compound (I) and a pharmaceutically acceptable salt thereof to be used in the present invention have superior IL-2 inhibitory action and immunosuppressive action, antibacterial action and other pharmacological activity, so that they are useful for the prophylaxis and treatment of rejection in organ or tissue transplantation, graft versus host reaction, autoimmune diseases, infectious diseases and the like, as noted, together with the production method thereof, in, for example, EP-A-184162, EP-A-323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/5059 and the like, all of these publications are hereby incorporated by reference.

In particular, the compounds called FR900506 (=FK506), FR900520 (Ascomycin), FR900523 and FR900525 are produced by the genus Streptomyces, such as Streptomyces tsukubaensis, No. 9993 (depository: National Institute of Advanced Industrial Science and Technology, International Patent Organism Depositary, Central 6, 1-1 Higashi 1-chome, Tsukuba-shi, Ibaraki-ken, Japan (formerly: Fermentation Research Institute, Agency of Industrial Science and Technology, the Ministry of International Trade and Industry), date of deposit: Oct. 5, 1984, deposit number: FERM BP-927) or Streptomyces hygroscopicus subsp. Yakushimaensis, No. 7238 (depository: National Institute of Advanced Industrial Science and Technology, International Patent Organism Depositary, Central 6, 1-1 Higashi 1-chome, Tsukuba-shi, Ibaraki-ken, Japan (formerly: Fermentation Research Institute, Agency of Industrial Science and Technology, the Ministry of International Trade and Industry), date of deposit: Jan. 12, 1985, deposit number: FERM BP-928 (EP-A-0184162), and the compound of the following formula), FK50.6 (general name: Tacrolimus) is a representative compound.

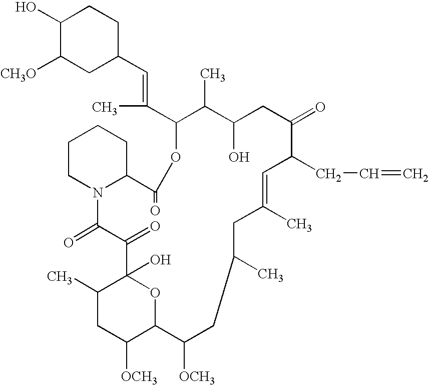

### Chemical Name: 17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo-[22.3.1.0^{4,9}]octacos-18-ene-2,3,10,16-tetraone

Of the tricyclo compounds (I), more preferred is a compound wherein adjacent pairs of R³ and R⁴, and R⁵ and R⁶ may each independently form another bond between carbon atoms binding with the members of each pairs;
R⁸ and R²³ each independently show hydrogen atom;
R⁹ is hydroxy;
R¹⁰ is methyl, ethyl, propyl or allyl;
X is (hydrogen atom, hydrogen atom) or oxo;
Y is oxo;
R¹⁴, R¹⁵, R¹⁶, R¹⁷, R¹⁸, R¹⁹ and R²² each independently show methyl;
R²⁴ is 3-R²⁰-4-R²¹-cyclohexyl,
   wherein R²⁰ is hydroxy, alkyloxy or —OCH₂OCH₂CH₂OCH₃, and
   R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy or R²⁵R²⁶CHCOO— (wherein R²⁵ is hydroxy optionally protected where desired, or protected amino, and R²⁶ is hydrogen atom or methyl), or R²⁰ and R²¹ in combination form an oxygen atom of epoxide ring; and
n is 1 or 2.

Particularly preferable tricyclo compound (I) include, besides FK506, Ascomycin derivatives such as halogenated derivative of 33-epi-chloro-33-desoxy Ascomycin described in Example 66a of EP-A-427,680 and the like.

Other preferable IL-2 inhibitors (macrolide compounds) include Rapamycin described in MERCK INDEX, 12 edition, No. 8288 and derivatives thereof. Preferable examples thereof include O-substituted derivative described at page 1 of WO95/16691, formula A, wherein the 40^{th} hydroxy is —OR₁ (wherein R₁ is hydroxyalkyl, hydroalkyloxyalkyl, acylaminoalkyl or aminoalkyl), such as 40-O-(2-hydroxy)ethyl Rapamycin, 40-O-(3-hydroxy)propyl Rapamycin, 40-O-[2-(2-hydroxy)ethoxy]ethyl Rapamycin and 40-O-(2-acetaminoethyl)Rapamycin. These O-substituted derivatives can be produced by reacting, under appropriate conditions, Rapamycin (or dihydro or deoxo Rapamycin) and an organic radical bound with a leaving group (e.g., RX wherein R is an organic radical desirable as O-substituent, such as alkyl, allyl and benzyl moiety, and X is a leaving group such as CCl₃C(NH)O and CF₃SO₃)). The conditions are: when X is CCl₃C(NH)O, acidic or neutral conditions, such as in the presence of trifluoromethanesulfonic acid, camphorsulfonic acid, p-toluenesulfonic acid or their corresponding pyridinium or substituted pyridinium salt, and when X is CF₃SO₃, in the presence of a base such as pyridine, substituted pyridine, diisopropylethylamine and pentamethylpiperidine. The most preferable Rapamycin derivative is 40-O-(2-hydroxy)ethyl Rapamycin as disclosed in WO94/09010, which is hereby incorporated into the specification by reference.

The pharmaceutically acceptable salt of tricyclo compound (I), Rapamycin and derivatives thereof are nontoxic and pharmaceutically acceptable conventional salts, which are exemplified by salts with inorganic or organic base such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

In the IL-2 inhibitor of the present invention, particularly macrolide compound, conformers and one or more pairs of stereoisomers such as optical isomers and geometric isomers, which are due to asymmetric carbon atom and double bond, may be included. Such conformers and isomers are also encompassed in the present invention. In addition, macrolide compounds can form solvates, which case is also encompassed in the present invention. Preferable solvate is exemplified by hydrates and ethanolates.

Other IL-2 inhibitors are known from MERCK INDEX, 12^{th} ed., No. 2821, U.S. Pat. Nos. 4,117,118, 4,215,199, 4,288,431, 4,388,307, Helv. Chim. Acta, 60, 1568 (1977) and 65, 1655 (1982) and Transplant. Proc. 17, 1362 (1985) and the like. Specifically, they are cyclosporins such as cyclosporin A, B, C, D, E, F and G and derivatives thereof. Particularly preferred is cyclosporin A. These publications are hereby incorporated into the specification by reference.

The tricyclo compound (I), pharmaceutically acceptable salt thereof, cyclosporins and derivatives thereof can be classified as “IL-2 production inhibitor” that inhibits production of IL-2. Rapamycin and derivative thereof can be classified as “IL-2 signal transduction inhibitor” that inhibit transmission of IL-2 signal.

In the present invention, the allergic disease encompasses any reaction type of IgE dependent anaphylactic type (I type), cytotoxic type (II type), immune complex type (III type) and cellular immunity type (IV type), as classified by Coombs and Gell (1963) mentioned above. In particular, bronchial asthma, allergic rhinitis, allergic conjunctivitis, atopic dermatitis, food allergy, drug allergy and the like classified under I type allergy are the suitable diseases to be targeted.

The treatment in the context of the present invention includes any management such as prevention, cure, alleviation of symptom, reduction of symptom, prevention of progression and the like.

By “for pre-administration” of the “pharmaceutical agent for pre-administration” and “pharmaceutical composition for pre-administration” of the present invention is meant administration in advance in a treatment method of allergic diseases, which comprises administering an IL-2 inhibitor to a subject in need of a treatment of allergic diseases for a given period of time (i.e., leading period) and thereafter again administering an effective amount of an IL-2 inhibitor. In the present specification, an IL-2 inhibitor used for pre-administration is distinguished from an IL-2 inhibitor to be administered after the leading period for pre-administration, as an IL-2 inhibitor to be used for treatment of allergic disease (to be referred to simply as during treatment).

According to the present invention, by setting a leading period for pre-administration of an IL-2 inhibitor, the effect on the allergic diseases can be expressed in a remarkably enhanced manner. For example, since the period of onset and termination of seasonal allergic diseases are mostly determined, a leading period for pre-administration set before the probable season of the onset of the disease enables more effective treatment of the disease. In the present invention, therefore, seasonal allergic diseases such as seasonal allergic conjunctivitis and seasonal allergic rhinitis are among the suitable target diseases. In addition, by setting a leading period for pre-administration, treatment with an IL-2 inhibitor at lower concentrations or with less frequency of the instillation per day, when the allergic diseases can be treated, becomes attainable thereby decreasing the burden on the patient.

According to the present invention, the above-mentioned IL-2 inhibitor is administered in an effective amount for the treatment of allergic disease to a subject in need thereof, after the leading period for pre-administration.

The IL-2 inhibitor used in the present invention for pre-administration and/or treatment of allergic diseases can be used as a pharmaceutical agent for human and animals, and can be administered systemically or locally by oral administration, intravenous administration (inclusive of transfusion), subcutaneous administration, rectal or virginal administration, administration to a local site of the eye (inclusive of eye ointment), administration to a local site of the nose (inclusive of spray). In consideration of systemic influence, significant expression of the effect and like, it is particularly preferably used in a form suitable for local administration.

The dosage form may be, for example, eye drop, eye ointment, nasal drop, spray, powder, granule, tablet, capsule, injection, ointment and the like, with particular preference given to eye drop, eye ointment, nasal drop, spray and the like. Such preparations can be produced according to conventional methods.

In the present invention, the leading period for pre-administration of an IL-2 inhibitor varies depending on the kind, age, body weight, condition to be treated, desired therapeutic effect, administration route and the like of the subject to be treated, such as human and animal. In general, the period is from 3 days to about 2 months, preferably from about 1 week to 1 month, which is determined as appropriate.

The dose of the IL-2 inhibitor during the leading period varies depending on the kind, age, body weight, condition to be treated, desired therapeutic-effect, administration route, treatment period, leading period, and the like, with regard to the subject to be treated, such as human and animal. Generally, when it is administrated systemically, the dose is about 0.0001-1000 mg, preferably 0.001-500 mg, which is given in a single dose or 2 to 4 dividual doses a day or in a sustained manner. When it is administered locally to the eye, a preparation containing the active ingredient in a proportion of 0.001-10.0 w/v %, preferably 0.005-5.0 w/v %, is applied several times a day per eye, preferably instilled or applied 1 to 6 times a day.

After the leading period for pre-administration of an IL-2 inhibitor, the dose and administration frequency of the IL-2 inhibitor for the treatment of the allergic disease are within the range specified above for the leading period. According to the present invention, the presence of the leading period enables reduction of the dose and administration frequency of the IL-2-inhibitor during the treatment.

The kind of the IL-2 inhibitor to be administered during the treatment is appropriately determined depending on the condition to be treated, desired therapeutic effect, administration route, treatment period, leading period and the like. It is preferable that the same IL-2 inhibitor administered during the leading period be used.

The present invention is explained in more detail in the following by way of Examples. The present invention is not limited by these Examples in any way.

### EXAMPLES

### Experimental Example 1

Method 1

Patients with allergic conjunctivitis (3 groups, 30 patients per group) were instilled with an antigen into the eye. Three minutes later, itchiness of the eye was evaluated in 9 levels (0, 0.5, 1, 1.5, 2, 2.5, 3, 3.5, 4), and the average value (base line group) of itchiness was determined. A specific antigen was determined for each patient. The antigen determined was the one against which the patient showed highest sensitivity in the preceding confirmation test using 9 kinds of antigens (cat hair, cat dander, ragweed, birch, oak, maple, meadow fescue, rye, kentucky blue).

Method 2

At least one week was allowed to lapse from the test of Method 1, and FK506 eye drop (suspension) [0.03%, 0.06% or 0.1%] was instilled into the eye of the patients. At 8 hr after the instillation of the eye drop, the antigen was given. Three minutes later, itchiness of the eye was evaluated in the same manner as in Method 1, and the average value (without pre-administration) of itchiness was determined. In the group without pre-administration, the proportion (improvement rate) of the patients who showed at least 1 point lower itchiness than the base line itchiness was determined. The results are shown in Table 1.

Method 3

At least one week was allowed to lapse from the test of Method 2, and FK506 eye drop was instilled into the eye of the patients once a day for one week. At 16 hr after the last instillation of the eye drop, the antigen was given. Three minutes later, itchiness of the eye was evaluated in the same manner as in Method 1, and the average value (with pre-administration) of itchiness was determined. In the group with pre-administration, the proportion (improvement rate) of the patients who showed at least 1 point lower itchiness than the base line itchiness was determined. The results are shown in Table 1.

TABLE 1

| | improvement (%) of | improvement (%) of |
|---|---|---|
| Drug con- | group without | group with |
| centration | pre-administration | pre-administration |
| 0.03% | 25.93 | 61.54 |
| 0.06% | 40.00 | 65.52 |
| 0.1% | 56.67 | 72.41 |

### INDUSTRIAL APPLICABILITY

From the above results, it is evident that the improvement effect on the itchiness can be strikingly enhanced by setting a leading period for pre-administration of FK506. For example, the improvement rate of the 0.03% concentration group with pre-administration was higher than that of the 0.1% concentration group without pre-administration. Hence, by setting a leading period for pre-administration, the allergic disease can be treated with a lower concentration of a medicament. In the comparison of 0.1% concentration groups, the improvement rate of the group with pre-administration after 16 hr was markedly higher than that of the group without pre-administration after 8 hr. Hence, by setting a leading period for pre-administration, the allergic disease can be treated with less frequency of the instillation.

1-18. (canceled)

19. A method for treating an allergic disease, which comprises pre-administering an interleukin 2 inhibitor for a leading period and then administering an effective amount of an interleukin 2 inhibitor to a subject in need of a treatment of an allergic disease.

20. The method of claim 19, wherein the interleukin 2 inhibitor is a macrolide compound or a cyclosporin.

21. The method of claim 20, wherein the macrolide compound is a tricyclo compound (I) of the following formula; 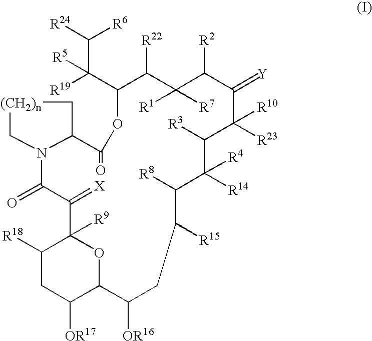
wherein adjacent pairs of R¹ and R², R³ and R⁴, and R⁵ and R⁶ each independently
a) consist of two adjacent hydrogen atoms, wherein R² is optionally alkyl, or
b) form another bond between carbon atoms binding with the members of each pairs;
R⁷ is hydrogen atom, hydroxy, alkyloxy or protected hydroxy, or may form oxo with
R⁸ and R⁹ each independently show hydrogen atom or hydroxy;
R¹⁰ is hydrogen atom, alkyl, alkenyl, alkyl substituted by one or more hydroxy, alkenyl substituted by one or more hydroxy, or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R ¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2; and
Y, R¹⁰ and R²³ may form, together with the carbon atom they bind with, a saturated or unsaturated 5 or 6-membered heterocyclic group containing nitrogen atom, sulfur atom and/or oxygen atom, wherein the heterocyclic group may be substituted by one or more group(s) selected from the group consisting of alkyl, hydroxy, alkyloxy, benzyl, a group of the formula —CH₂Se(C₆H₅), and alkyl substituted by one or more hydroxy,
or a pharmaceutically acceptable salt thereof.

22. The method of claim 20, wherein the macrolide compound is FK506.

23. The method of clam 19, wherein the interleukin 2 inhibitor is a preparation for local administration.

24. The method of clam 23, wherein the local administration is administration to the eye or to the nose.

25. The method of claim 19, wherein the allergic disease is allergic conjunctivitis.

26. The method of claim 19, wherein the allergic disease is seasonal allergic disease.

27. The method of claim 26, wherein the seasonal allergic disease is seasonal allergic conjunctivitis.

28-37. (canceled)

38. The method of claim 21, wherein the macrolide compound is FK506.
